(19) United States
(12) Patent Application Publication
(10) Pub. No.: US 2025/0195115 A1
(43) Pub. Date: Jun. 19, 2025

(54) SYSTEMS AND METHODS TO FUSE BONE

(71) Applicant: THORAGENIX INNOVATIONS, INC., Boca Raton, FL (US)

(72) Inventors: Frederick J. THABET, Boca Raton, FL (US); Ravi KANAGALA, Boca Raton, FL (US); Keri GEORGE, Calgary (CA)

(73) Assignee: THORAGENIX INNOVATIONS, INC., Boca Raton, FL (US)

(21) Appl. No.: 18/849,778

(22) PCT Filed: Mar. 22, 2023
(86) PCT No.: PCT/US2023/015947

§ 371 Date: Sep. 23, 2024

Related U.S. Application Data
(60) Provisional application No. 63/323,371, filed on Mar. 24, 2022.

(51) Int. Cl. A61B 17/80

(52) U.S. Cl. CPC A61B 17/8076; A61B 17/808; A61B 17/809

(57) ABSTRACT

A system for fusing two sections of bone includes an implant, a bone clamp, and/or a cancellous bone manipulator tool. The implant, bone clamp, and cancellous bone manipulator tool can form an integrated medical kit. The implant has geometric protrusions extending from a first side and a second side to form bone anchors that mate with the cancellous tissue of the sections of bone. Additionally, the bone clamp has a single-handed handle arrangement for opening and closing the bone clamp with one hand. For instance, a first handle of the bone clamp extends from a shaft and a second handle of the bone clamp is spaced apart from the first handle and extends from a spring-loaded slider slidably coupled to the shaft. Upon compressing and securing the two sections of bone and the implant together, a carrier component can be introduced into the implant through a preformed port.

### CROSS-REFERENCE TO RELATED APPLICATION

This application claims priority to U.S. Provisional Patent Application No. 63/323,371, filed Mar. 24, 2022, the entirety of which is herein incorporated by reference.

### BACKGROUND

### 1. Field of the Invention

The presently disclosed technology relates to systems and methods to heal and/or replace bone. In at least one example, the presently disclosed technology relates to an implant system and method to stabilize and fuse two halves of a sternum of a patient post-sternotomy.

### 2. Description of Related Art

The sternum is a long, flat bone forming the middle portion of the front of the chest. Individual rib bones are connected along the sides of the sternum via cartilage to form the ribcage which protects the heart, lungs, and major blood vessels from injury. In a sternotomy, a surgeon cuts open the sternum to gain access to the thoracic contents to perform cardiothoracic surgery. A midline longitudinal incision is made through at least a portion of the sternum to allow opposing halves to be laterally separated to provide access to organs within the ribcage. When the surgical procedure is complete, the separated halves are aligned with and secured to one another and the incision is closed.

The ideal goal for closing and healing sternotomy wounds is complete rejoining of the sternal halves with new bone growth in the absence of any complications. Unfortunately, patient recovery from a conventional sternotomy is often slow and problematic. As the two sections of the sternum are brought back together by the surgeon, proper compression and a tight realignment of the end plates of the sternal surfaces is rarely achieved, resulting in non-union of the separated sternal halves. This non-union allows for motion such as sliding of the surface of one sternal half against the surface of the other sternal half, which leads to significant pain for the patient and increased chance for development of infection. Additionally, the lack of compression leads to excessive bleeding from the sternal edge. This results in significant post-operative blood loss which further leads to lengthy intensive care unit (ICU) and hospital stay times. In addition, due to the significant post-operative blood loss, physicians are forced to keep the intubation tube in place so that if the patient needs to be taken back into surgery to control the bleeding coming from the sternal edge, they can do so quickly without having to re-intubate the patient. As a further complication, these extended intubation periods can lead the patient to develop pneumonia.

Furthermore, the lack of proper compression can lead to the formation of fibrous scar tissue instead of the desired new bone. The building up of scar is problematic as well, especially in situations in which further surgical intervention is necessary. The scar tissue often adheres to internal structures in addition to the sternum, such as cardiac blood vessels. If a surgeon needs to conduct additional surgery, the surgeon must first remove scar tissue covering the vessels. If the scar tissue is prevalent enough, the surgeon could easily cause injury to or even death of the patient during subsequent surgeries.

Another serious complication associated with sternotomy is the development of a deep sternal wound infection (DSWI), particularly within spaces left due to dehiscence of the two sections of the sternum. DSWI has up to a 6% incidence of occurrence after cardiac surgery and a high mortality rate, which prolongs hospital stay and significantly increases cost of care (e.g., up to $450,000).

Separation procedures for other types of bones suffer similar problems when attempting to rejoin the separated bone portions. It is with these observations in mind, among others, that various aspects of the present disclosure were conceived and developed.

### SUMMARY

The presently disclosed technology addresses the foregoing problems by providing systems and methods for fusing two sections of bone. For instance, a system to fuse two sections of a bone can comprise: an at least partially synthetic implant including: an elongated body; first one or more bone anchors at a first side of the elongated body; and second one or more bone anchors at a second side of the elongated body; and a bone clamp with a single-handed handle arrangement.

The system can further comprise an elongated cancellous bone manipulator tool with a curved paddle at a first manipulator end and a scraper surface at a second manipulator end. The at least partially synthetic implant, the bone clamp, and the elongated cancellous bone manipulator tool can form an integrated medical kit. Moreover, the single-handed handle arrangement can include: a shaft defining a length of the bone clamp with a first handle extending from the shaft; and a spring-loaded slider slidably coupled to the shaft with a second handle extending from the spring-loaded slider. In some instances, the first handle extends from a first shaft end, the second handle extends from a first slider end, and the bone clamp further includes, a first gripping arm extending from a second shaft end opposite the first shaft end; and a second gripping arm extending from a second slider end opposite the first slider end.

In some examples, the first gripping arm and the second gripping arm are maintained in a closed position by a spring when the bone clamp is in a resting state. Furthermore, the first one or more bone anchors and the second one or more bone anchors are a plurality of geometric protrusions operable to mate with cancellous tissue of the two sections of bone. The plurality of geometric protrusions can have a triangular lateral cross-section profile and a trapezoidal longitudinal cross-section profile. Additionally, the elongated body can include a plurality of implant sections with a plurality of length dimensions corresponding to a plurality of particular bones. For instance, the plurality of particular bones can include a manubrium, a sternum body, and a xiphoid process.

In some instances, a system to fuse two sections of a bone comprises: an at least partially synthetic implant including: an elongated body; a first bone anchor extending from a first side of the elongated body as a first geometric protrusion; and a second bone anchor extending from a second side of the elongated body as a second geometric protrusion; and a bone clamp to compress the at least partially synthetic implant between the two sections of bone.

The first geometric protrusion can be shaped to press into first cancellous tissue of a first section of the two sections of the bone, and the second geometric protrusion can be shaped to press into second cancellous tissue of a second section of the two sections of bone. Furthermore, the bone clamp can have a single-handed handle arrangement including: a shaft with a spring-loaded slider slidably coupled to the shaft, a first handle extending from the shaft, and a second handle extending from the spring-loaded slider. The at least partially synthetic implant can be formed of a porous and/or fibrous material operable to receive a carrier component, and the at least partially synthetic implant has a port to receive the carrier component, the port being disposed on a front surface of the elongated body between the first side and the second side. Additionally, in some instances, the port includes a tactile identifier. The tactile identifier can be a raised or serrated lip around the port.

In some examples, a method to fuse two sections of a bone comprises: forming an impression in a cancellous tissue portion of the two sections using an end of a cancellous bone manipulator tool; disposing an at least partially synthetic implant between the two sections of the bone, the at least partially synthetic implant including one or more protruding bone anchors to mate with cancellous tissue of the two sections of the bone; and compressing the at least partially synthetic implant between the two sections using a bone clamp with a single-handed handle arrangement. The single-handed handle arrangement can include a first handle spaced apart from a second handle, the first handle extending from a shaft of the bone clamp, and the second handle extending from a spring-loaded slider of the bone clamp slidably coupled to the shaft, and using the bone clamp includes pulling the first handle towards the second handle.

Additionally, the method can further include securing the at least partially synthetic implant and the two sections of bone together, and introducing, subsequent to securing the at least partially synthetic implant and the two sections of bone together, a carrier component into porous and/or fibrous material forming the at least partially synthetic implant. In some instances, introducing the carrier component into the porous and/or fibrous material includes introducing the carrier component through a preformed port disposed on the at least partially synthetic implant.

The foregoing is intended to be illustrative and is not meant in a limiting sense. Many features of the embodiments may be employed with or without reference to other features of any of the embodiments. Additional aspects, advantages, and/or utilities of the presently disclosed technology will be set forth in part in the description that follows and, in part, will be apparent from the description, or may be learned by practice of the presently disclosed technology.

### BRIEF DESCRIPTION OF THE DRAWINGS

The foregoing summary, as well as the following detailed description, will be better understood when read in conjunction with the appended drawings. For the purpose of illustration, there is shown in the drawings certain embodiments of the disclosed subject matter. It should be understood, however, that the disclosed subject matter is not limited to the precise embodiments and features shown. The accompanying drawings, which are incorporated in and constitute a part of this specification, illustrate an implementation of systems and methods consistent with the disclosed subject matter and, together with the description, serves to explain advantages and principles consistent with the disclosed subject matter, in which:

FIG. 1 illustrates an example system including various tools of a bone fusing medical kit for fusing two sections of bone;

FIG. 2 illustrates an example system including an at least partially synthetic implant, which can form at least a portion of the system of FIG. 1;

FIG. 3 illustrates an example system including an at least partially synthetic implant with one or more ports, which can form at least a portion of the system of FIG. 1;

FIG. 4 illustrates an example system including an at least partially synthetic implant with one or more ports, which can form at least a portion of the system of FIG. 1;

FIG. 5 illustrates an example system including an at least partially synthetic implant with a pinched portion and one or more flared portions, which can form at least a portion of the system of FIG. 1;

FIG. 6 illustrates an example system including an at least partially synthetic implant having a plurality of implant segments, which can form at least a portion of the system of FIG. 1;

FIG. 7 illustrates an example system including one or more ports, which can form at least a portion of the system of FIG. 1;

FIG. 8 illustrates an example system including a bone clamp with a single-handed handle arrangement, which can form at least a portion of the system of FIG. 1;

FIG. 9 illustrates an example system including a cancellous bone manipulator tool, which can form at least a portion of the system of FIG. 1;

FIG. 10 illustrates an example system including a bone fusing medical kit, which can form at least a portion of the system of FIG. 1; and

FIG. 11 illustrates an example method for fusing two sections of bone with a bone fusing medical kit, which can be performed by at least the system of FIG. 1.

### DETAILED DESCRIPTION

The following detailed description references the accompanying drawing that illustrates various embodiments of the presently disclosed technology. The illustration and description are intended to describe aspects and embodiments of the presently disclosed technology in sufficient detail to enable those skilled in the art to practice the presently disclosed technology. Other components can be used and changes can be made without deviating from the scope of the presently disclosed technology. The following detailed description is, therefore, not to be taken in a limiting sense. The scope of the presently disclosed technology is defined only by the appended claims, along with the full scope of equivalents to which such claims are entitled.

### I. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the presently disclosed technology or the appended claims. Further, it should be understood that any one of the features of the presently disclosed technology may be used separately or in combination with other features. Other systems, methods, features, and advantages of the presently disclosed technology will be, or become, apparent to one with skill in the art upon examination of the figures and the detailed description. It is intended that all such additional systems, methods, features, and advantages be included within this description, be within the scope of the presently disclosed technology, and be protected by the accompanying claims.

The term “implant” or “surgical implant” can refer to a medical device manufactured to replace a missing biological structure, support a damaged biological structure, or enhance an existing biological structure.

The term “sternotomy” can refer to the surgical procedure of cutting a patient's sternum and separating cut portions to access thoracic organs such as the heart, lungs, and/or blood vessels. While the disclosed subject matter is focused on the sternum, it is foreseen that the system and method can be utilized in relation to any separated portions of any bone.

The term “patient” can include any human being or animal. The term “subject” may also be used herein to refer to the patient.

Cancellous bone can be a meshwork of spongy tissue of mature bone, for example found at the core of vertebral bones in the spine and/or at the ends of long bones such as the femur.

The term “bone growth-promoting material” can include any material that promotes and/or enhances bone growth both natural and synthetic. The term “bone growth-promoting agent” can include any composition or material that promotes and/or enhances bone growth. The bone growth-promoting agent added to the implant can be any known bone-growth promoting agent, including, but not limited to hydroxyapatite (HA), cellular growth factors, cytokines, silicates, and bone morphogenetic proteins (BMP). In some examples, strips of bone growth-promoting material, for example cancellous bone, fiber bone and/or collagen sponge, include one or more types of living cells. Living cells can also be carried by synthetic bone materials intended for the same purpose. The living cells added to the implant can be any living cells that promote and/or enhance bone growth including, but not limited to, stem cells, osteoblasts, osteoconductive cells, osteoinductive cells, and/or osteogenic cells. In some examples, strips of bone growth-promoting material, for example cancellous bone, fiber bone, synthetics, and/or collagen sponge, can include both bone growth-promoting agents and living cells as described herein.

The term “synthetic” material can include any man-made material. The synthetic material can be made from a combination of natural material and/or man-made or fabricated material.

Further, any term of degree such as, but not limited to, “substantially,” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 1 mm includes all values from 0.1 mm to 9 mm. Additionally, the term “about” can refer to near or close to the desired dimension, for example “about” can refer to near or close to disclosed thicknesses and encompassed thicknesses that can be effectively implanted into the patient.

Further, as the presently disclosed technology is susceptible to embodiments of many different forms, it is intended that the present disclosure be considered as an example of the principles of the presently disclosed technology and not intended to limit the presently disclosed technology to the specific embodiments shown and described. Any one of the features of the presently disclosed technology may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments, but is not necessarily included. Thus, the presently disclosed technology may include a variety of combinations and/or integrations of the embodiments described herein. Additionally, all aspects of the present disclosure, as described herein, are not essential for its practice. Likewise, other systems, methods, features, and advantages of the presently disclosed technology will be, or become, apparent to one with skill in the art upon examination of the figures and the description. It is intended that all such additional systems, methods, features, and advantages be included within this description, be within the scope of the presently disclosed technology, and be encompassed by the claims.

The term “coupled” is defined as connected, whether directly or indirectly through intervening components, and is not necessarily limited to physical connections. The connection can be such that the objects are permanently connected or releasably connected. The terms “comprising,” “including” and “having” are used interchangeably in this disclosure. The terms “comprising,” “including” and “having” mean to include, but not necessarily be limited to the things so described.

Lastly, the terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

### II. General Architecture

Systems and methods disclosed herein improve upon previous techniques for fusing two sections of bone. The systems include various tools forming a bone fusing medical kit that, when used together or individually, dramatically improve patient outcomes for a post-sternotomy bone fusing procedure. For instance, the systems can include a cancellous bone manipulator tool for preparing the cut portions of the bone sections, an at least partially synthetic implant (“implant”) for mating the cut portions of the two sections of bone together, and a bone clamp for compressing the implant between the two sections of bone. After securing the compressed two sections of bone and the implant together with wires, a carrier component (e.g., anesthesia, bone growth-promoting material, medications, and the like) can be injected into the implant through a port disposed on a front surface of the implant. The port can include a tactile identifier, such as a raised lip, to help the surgeon find the port without requiring a visual line-of-sight.

The implant can include an elongated body with bone anchors extending from both sides. The bone anchors can include geometric protrusions of fibrous/porous material with shapes configured to mate with the cancellous bone tissue of the two sections of bone. Additionally, the bone clamp includes a single-handed handle arrangement. The various components of the bone clamp are arranged so the bone clamp is in a spring-loaded, closed position when in a resting state (e.g., not being actuated), and can be moved to an open position with a single hand. A first handle extending from a shaft of the bone clamp can be spaced a distance apart from a second handle extending from a spring-loaded slider coupled to the shaft. Pulling the second handle towards the first handle causes the spring-loaded slider to move in a linear direction parallel to the shaft, pulling the bone clamp open. Releasing the second handle from the first handle (e.g., once the bone clamp is positioned around the two bone sections and the implant) causes the spring to push the bone clamp closed.

The compressed and secured implant acts like a gasket and can accomplish multiple benefits, such as hemostasis, pain relief, realignment, setting the bone to fuse and heal versus stabilizing via soft tissue or scar tissue formation, stabilizing the entire sternal construct, and/or eliminating gapping and loosening of the sternal construct with wires. By reducing or eliminating the gaps that may form, the areas where bacteria can form and create infection are reduced or eliminated. Moreover, antibiotics, growth agents, pain medications, and other carrier components injected into the port further reduce infections.

The system can provide sternal reinforcement and reconstruction. For instance, the sternal bone may be compromised by one or more factors such as trauma, disease, osteoporosis, age, infection, iatrogenic compromise, obesity, diabetes, or other co-morbidities. The implant can rebuild and reconstruct the integrity of a compromised sternum. Moreover, the systems disclosed herein can reconstruct a pediatric sternum and provide proper healing to improve the long-term health of pediatric patients. The system can further act as a pain reduction platform to help reduce post-operative pain caused by coughing and movement. Since the chest wall moves when a patient coughs post-operatively, the ragged edges of the incised bone translate against themselves. This translation can cause exposed and raw nerve endings to become severely irritated and can produce massive amounts of pain. The implant can cushion, shield, and protect the exposed nerve endings and buffer the translational forces to significantly reduce post-operative pain.

This results in significantly improved patient outcomes and reduced costs for the patient and hospital alike. Additional advantages of the technology will become apparent from the disclosure herein.

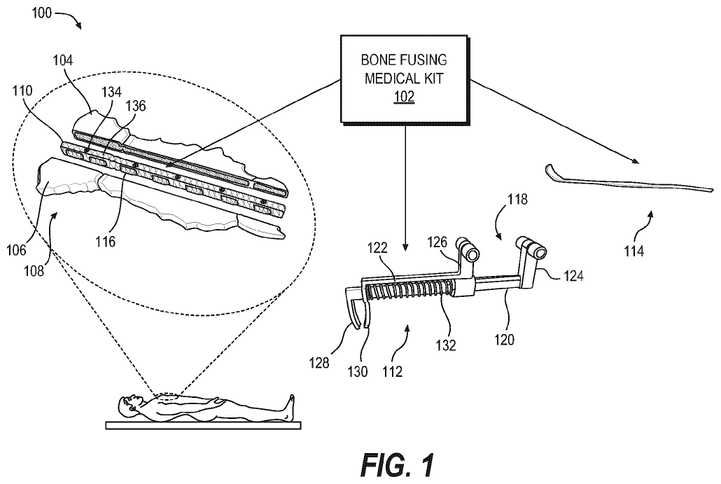

FIG. 1 is an example diagram of a system 100 including a bone fusing medical kit 102 to fuse two separated sections of bone. The bone fusing medical kit 102 can include various tools for fusing a first bone section 104 (e.g., a first half) a second bone section 106 (e.g., a second half) of a sternum 108. For instance, the system 100 may be used after performing an operation on a heart of a patient (e.g., in an operating room environment). The various tools of the bone fusing medical kit 102 can include an at least partially synthetic implant or “implant” 110, a bone clamp 112, and/or a cancellous bone manipulator tool 114. A surgeon can use the various tools at different stages of a bone fusing procedure.

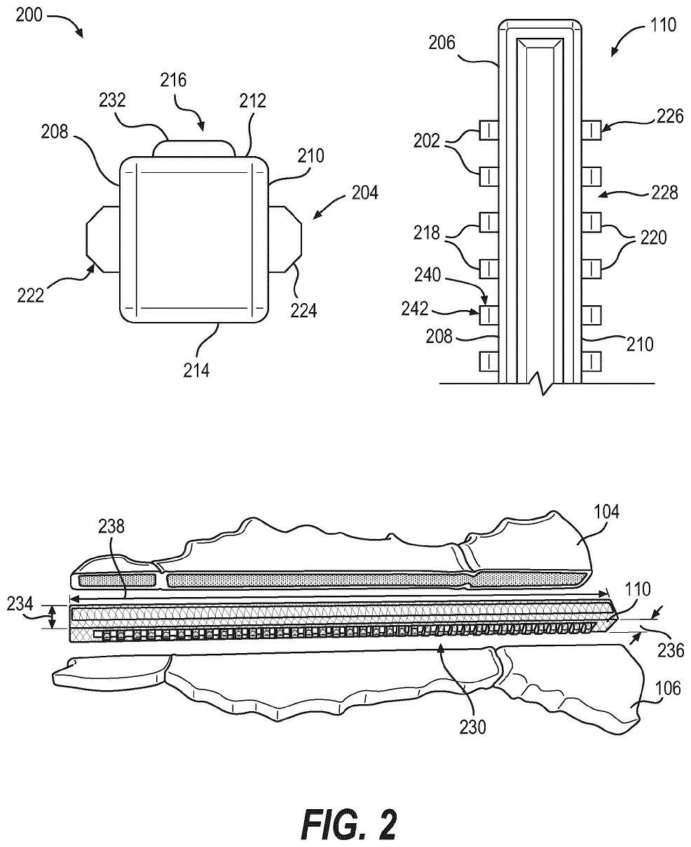

For instance, the cancellous bone manipulator tool 114 can be used to form one or more impressions in cancellous tissue of the first bone section 104 and/or the second bone section 106 during a preparation process. The cancellous tissue can be shaped and/or roughened with the cancellous bone manipulator tool 114 to prepare the cancellous tissue to receive the implant 110. In some examples, the implant 110 can have one or more bone anchors 116 protruding from sides of the implant 110, as discussed in greater detail below regarding FIGS. 2-6. The one or more bone anchors 116 can have a geometric shape (e.g., a pyramid, an ovoid, a rectangular prism, etc.) for mating with corresponding impression formed into the cancellous tissue of the first bone section 104 and/or the second bone section 106.

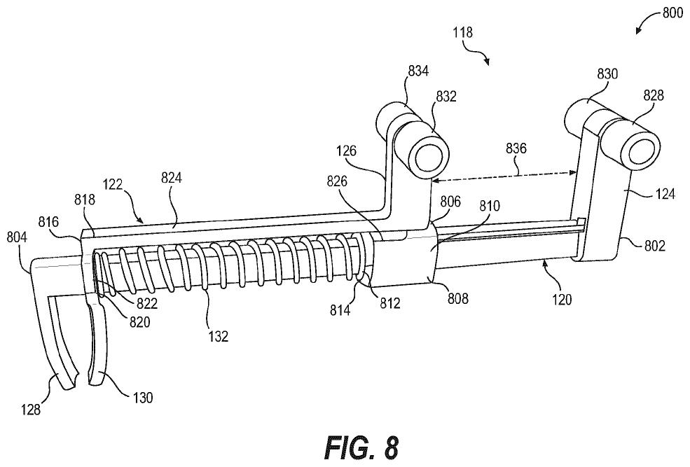

In some examples, a bone compression process can be performed with the bone clamp 112. Upon preparing the cancellous tissue with the cancellous bone manipulator tool 114 and positioning the implant 110 between the prepared first bone section 104 and second bone section 106, the bone clamp 112 can compress the first bone section 104, the second bone section 106, and the implant 110 together, for instance, using a single-handed handle arrangement 118. The single-handed handle arrangement 118 can include a shaft 120 with a spring-loaded slider 122 slidably coupled to the shaft 120, a first handle 124 extending from the shaft 120, and a second handle 126 extending form the spring-loaded slider 122. As discussed below regarding FIG. 8, the single-handed handle arrangement 118 can further include a spacing distance between the first handle 124 and second handle 126, perpendicular rollers extending from the first handle 124 and the second handle 126, and/or a closed resting position. Accordingly, a surgeon or other medical personnel can use the single-handed handle arrangement 118 to pull the bone clamp 112 open with one hand while the other hand is holding the implant 110 in place and position the bone clamp 112 around the separated sternum. With a first gripping arm 128 abutting the first bone section 104 and a second gripping arm 130 abutting the second bone section 106, the surgeon closes the bone clamp 112 by releasing the first handle 124 from the second handle 126 and letting a spring 132 push the spring-loaded slider 122 along the shaft 120 so the first gripping arm 128 and the second gripping arm 130 are brought together.

In some examples, the first bone section 104, the implant 110, and the second bone section 106 can be secured together (e.g., with the implant 110 between the first bone section 104 and the second bone section 106) by one or more wires or fasteners (e.g., hooks, staples, screws, barbs, plates, etc.).

After securing the compressed first bone section 104, implant 110, and second bone section 106 together (e.g., in a sandwich configuration), a component carrier can be added to the implant 110. The component carrier can be introduced through one or more ports 134 located on a surface of the implant 110, such as a front surface 136. The implant 110 can be made of porous and/or fibrous material to receive the component carrier(s), which can be one or more of a cellular growth factor such as bone morphogenetic proteins, mesenchymal stem cells, blood, osteoclasts, osteoblasts, antibiotics, analgesics, medications, and the like. In some instances, the one or more ports 134 include a tactile indicator to provide tactile (e.g., physical or haptic) feedback when touched by the surgeon.

In some examples, the techniques disclosed herein for using the tools of the medical kit 102 (individually and/or in combination) result in compression across the sternal plane creating a successful fusion. The implant 110 can fill any voids or gaps caused by the sternal saw (e.g., by filling impressions formed by the cancellous bone manipulator tool 114), and proper alignment of the implant 110 with the first bone section 104 and the second bone section 106 results from using the bone clamp 112 with the single-handed handle arrangement 118 to free up the other hand for holding the implant in proper alignment. Moreover, the spring-loaded slider 122 of the bone clamp 112 can be configured to provide an amount of compression force appropriate for particular types of bone (e.g., a particular amount of compression force or pressure for fusing the sternum sections). As such, the system 100 disclosed herein can achieve optimal clinical results by significantly reducing out patient hospitalization time (e.g., to 6-8 hours), reducing risk of infection, and reducing costs to the hospital and patient alike.

Turning to FIGS. 2-6, multiple examples of the implant 110 are shown having various shapes, features, profiles, and bone anchors 116. For instance, FIG. 2 illustrates an example system 200 to fuse bone sections includes the implant 110 with a plurality of geometric protrusions 202 having a chamfered edge configuration 204.

The implant 110 can have an elongated body 206 with a first side 208, a second side 210, a front side 212, and a rear side 214 (e.g., forming a substantial rectangular top view cross section 216), which can generally comprise planar surfaces. The one or more bone anchors 116 can extend from any of the sides of the implant 110. For instance, the one or more bone anchors 116 include a first set of geometric protrusions 218 extending from the first side 208 and a second set of geometric protrusions 220 extending from the second side 210. The geometric protrusions 202 can have a variety of different three-dimensional shapes, geometries, or profiles. For instance, the geometric protrusions 202 (bone anchors 116) can have a chamfered top profile 222 including a square, rectangle, or other shape with one or more chamfered edges 224. Moreover, the geometric protrusions 202 can have a substantially rectangular or square front profile 226. In some instances, the geometric protrusions 202 can have a spacing distance 228 between each other and/or a substantially even distribution 230 along the first side 208 or the second side 210 (e.g., with a substantially same spacing distance between the geometric protrusions 202).

In some examples, a shape, size, profile, and/or distribution of the one or more bone anchors 116 can correspond to a particular size or shape of a particular bone for which the implant 110 is configured to fuse. For instance, the implant 110 can include between 30 and 70 bone anchors 116 with the substantially even distribution 230 for fusing sections of the sternum 108.

In some instances, the elongated body 206 can include a raised surface 232 along the front side 212. Moreover, the elongated body 206 can have a width dimension 234, a depth dimension 236, and/or a length dimension 238 configured for fusing the sections of the sternum 108. For instance, the width dimension 234 can be between 7 mm and 12 mm (e.g., 9 mm), the depth dimension 236 can be between 8 mm and 15 mm (e.g., 13 mm), and the length dimension 238 can be between 190 mm and 210 mm (e.g., 204 mm). Additionally, the one or more bone anchors 116 can have a width dimension 240 of between 2 mm and 3.5 mm (e.g., 2 mm, 2.7 mm, or 2.9 mm) and a length dimension 242 of between 2 mm and 20 mm (e.g., 2 mm, 17 mm, or 15.5 mm).

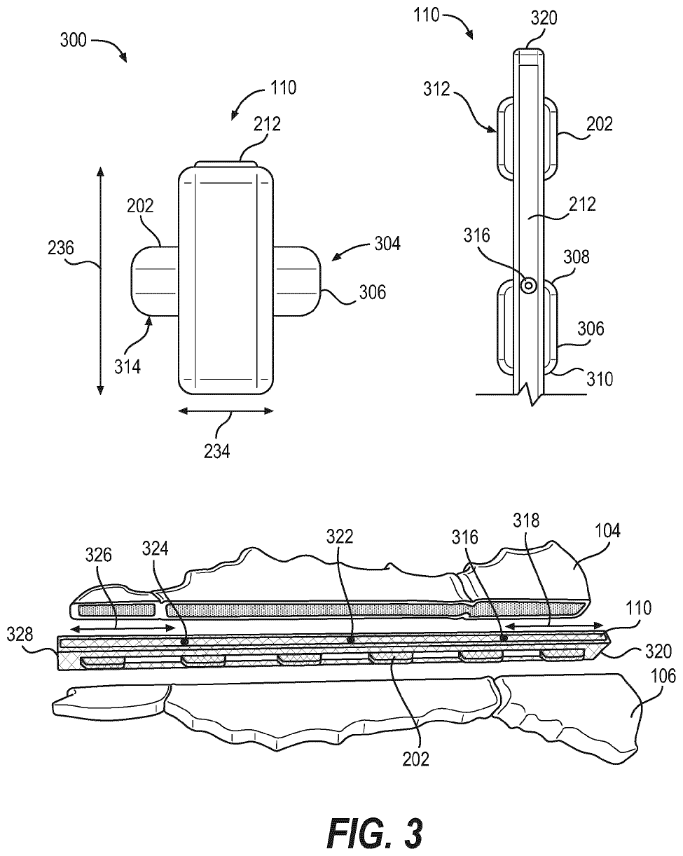

FIG. 3 illustrates an example system 300 to fuse bone sections using the implant 110 which, as depicted in FIG. 3, can have the plurality of geometric protrusions 202 with a rounded edge configuration 304. Moreover, FIG. 3 illustrates the implant 110 with the one or more ports 134 for receiving the carrier compound into the elongated body 206.

In some examples, one or more of the geometric protrusions 202 can have a protrusion side surface 306 that transitions into a rounded top end 308 and a rounded bottom end 310. The elongated body 206 of the implant 110 can have the depth dimension 236 being greater than the width dimension 234 (e.g., with between a 2:1 ratio and a 5:1 ratio) such that the implant 110 has a substantially rectangular top view cross section 216. The implant 110 depicted in FIG. 3 can have between two and ten geometric protrusions 202 (e.g., six geometric protrusions 202) extending from the first side 208 and/or the second side 210. The geometric protrusions 202 can have a rounded-corner rectangular front profile 312 and/or a rounded corner top profile 314. Additionally, the geometric protrusions 202 can have the length dimension 242 that is greater than the width dimension 240. For instance, the length dimension 242 of the geometric protrusions 202 can have a ratio with respect to the width dimension 240 of between 2:1 and 10:1.

Additionally, or alternatively, the implant 110 can include the one or more ports 134. For instance, a first port 316 of the one or more ports 134 can be disposed on the front side 212 of the elongated body 206, for instance, spaced a first distance 318 from a first terminating end 320 of the elongated body 206. Moreover, the implant 110 can include a second port 322, a third port 324, and/or any number of ports 134. For instance, the second port 322 can be located at a center of the front side 212, and the third port 324 can be located a second distance 326 from a second terminating end 328 of the elongated body 206, such that the implant 110 can include three evenly or uniformly spaced apart ports 134 on the front side 212.

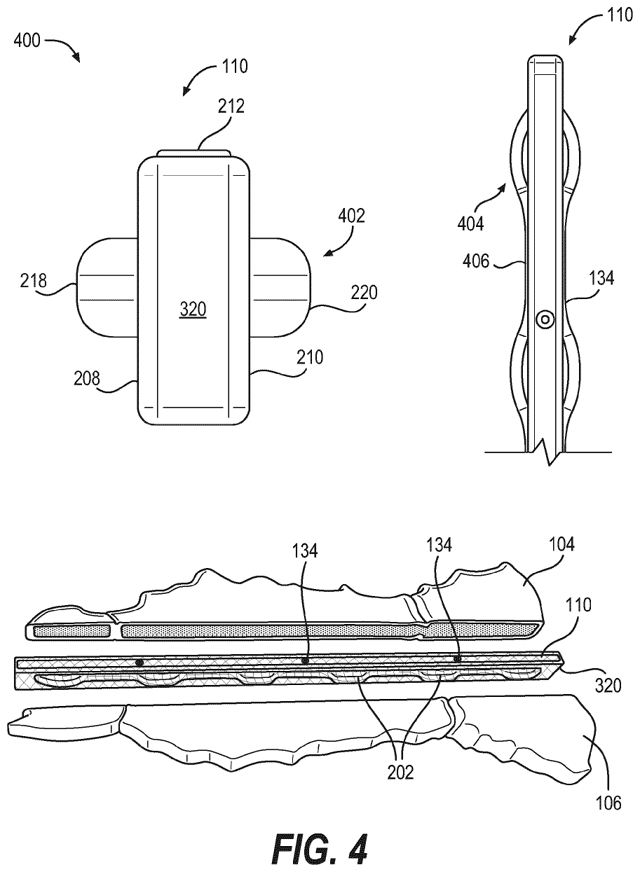

FIG. 4 illustrates an example system 400 to fuse bone sections using the implant 110 with the geometric protrusions 202 which, as depicted in FIG. 4, can have a partial ovoid or curved configuration 402. The partial ovoid or curved configuration 402 can be formed by the bone anchors 116 having a rounded “oscillating,” symmetrical wave, or sinusoidal side profile shape 404. The bone anchors 116 can include one or more flat gaps or spaces 406 between the sinusoidal side profile shapes 404 of the bone anchors 116. The partial ovoid or curved configuration 402 can include bone anchors 116 forming a curved or bent shape and/or shaped like a section of a disc protruding from the first side 208 and the second side 210.

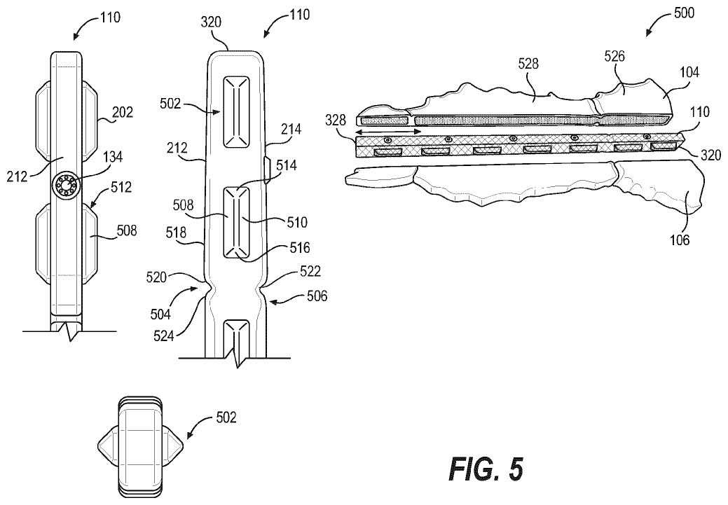

FIG. 5 illustrates an example system 500 to fuse bones sections using the implant 110 which, as depicted in FIG. 5, can have the geometric protrusions 202 with a trapezoidal pyramid configuration 502. Moreover, the implant 110 can include a pinched portion 504 (e.g., a narrowing portion) and/or one or more flared portions 506 (e.g., widening portions) along the elongated body 206.

In some examples, the geometric protrusions 202 can have a first slanted side 508 and a second slanted side 510 that adjoins the first slanted side 508 at a ridge. The first slanted side 508 and the second slanted side 510 can have a trapezoid-shaped surface 512 (e.g., which gives the geometric protrusions 202 a trapezoidal longitudinal cross-section profile). Additionally, the geometric protrusions 202 of the implant 110 in the trapezoidal pyramid configuration 502 can include a first slanted end 514 and a second slanted end 516 that adjoin the first slanted side 508 and the second slanted side 510. The first slanted end 514 and the second slanted end 516 can have triangle-shaped surfaces with a point terminating at the ridge (e.g., giving the geometric protrusions a triangular lateral cross-section profile). In some examples, the geometric protrusions 202 can omit the ridge such that the first slanted end 514 and the second slanted end 516 terminate at a same point, forming a pyramid with a rectangle or square base and four triangle sides (rather than two triangle sides and two trapezoidal sides).

In some instances, the elongated body 206 of the implant 110 includes the one or more flared portions 506. The implant 110 can have a first flared portion 518 including a depth dimension 236 that tapers, or becomes smaller from an intermediate point 520 on the elongated body 206 to the first terminating end 320. Moreover, the implant 110 can include the pinched portion 504 (e.g., transitioning from the intermediate point 520) including a narrowing or decreasing depth dimension 236 to form indents 522 in the front side 212 and the rear side 214. Transitioning from the pinched portion 504 along the length dimension 238 of the elongated body 206, the implant 110 can include a second flared portion 524. The second flared portion 524 can be a tapered depth dimension 236 extending from the pinched portion 504 along the elongated body 206 toward the second terminating end 328. The second flared portion 524 can transition to a consistent depth dimension 236 (e.g., with the front side 212 parallel to the rear side 214), and/or a smaller or narrower depth dimension 236 than that formed by the first flared portion 518. The pinched portion 504 and the one or more flared portions 506 can be substantially symmetrical and/or form a mirror image. Moreover, the implant 110 can include a port 134 (e.g., a single port) on the front side 212 at the first flared portion 518 and/or one or more ports 134 (e.g., a plurality of ports 134 such as four ports) on the front side 212 below the first flared portion 518.

In some instances, the pinched portion 504 and/or the one or more flared portions 506 (e.g., or other depth dimension features) can correspond to a shape or depth of the bone being fused. For example, the first flared portion 518, the pinched portion 504, and the second flared portion 524 correspond to or align with contours in the sternum 108 formed by a manubrium 526 and an upper portion of a sternum body 528 of the sternum 108.

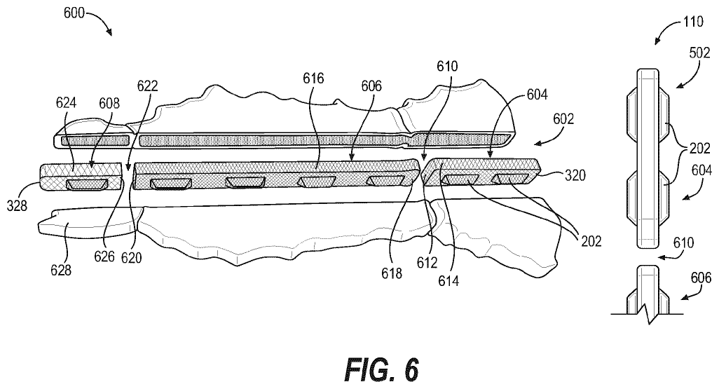

FIG. 6 depicts a system 200 including the implant 110 with the geometric protrusions 202 in the trapezoidal pyramid configuration 502 and a segmented elongated body 602. For instance, the implant 110 can include a first implant segment 604, a second implant segment 606, and a third implant segment 608.

The first implant segment 604 can include the first terminating end 320, the first flared portion 518, and at least a portion of the pinched portion 504. The implant 110 can include a first break 610 along the length dimension 238 at the indents 522. For instance, the implant 110 can have a preformed full first break and/or the implant 110 can have a preformed partial break (e.g., such as one or more holes, cuts, idents, perforations, and the like) so that the implant 110 can easily be broken, torn, or cut into the implant segments. Additionally or alternatively, the implant 110 can include a marking or indicator (e.g., such as a visual line) indicating where the first break 610 is to be formed. The first break 610 can define a third terminating end 612 forming the second distal end of the first implant segment 604 (e.g., with the first terminating end 320 forming the first distal end of the first implant segment 604). In some examples, the first implant segment 604 can have a first elongated body 614 with two geometric protrusions 202 extending from the first side 208 and two geometric protrusions 202 extending from the second side 210.

The second implant segment 606 can include a second elongated body 616 with a fourth terminating end 618 (e.g., a first distal end) defined by the first break 610 and a fifth terminating end 620 (e.g., a second distal end) defined by a second break 622. The second implant segment 606 can have the second elongated body 616 with four geometric protrusions 202 extending from the first side 208 and four geometric protrusions 202 extending from the second side 210. The second implant segment 606 can include more geometric protrusions 202 than the first implant segment 604 and/or the third implant segment 608. The second elongated body 616 can include at least a portion of the pinched portion 504 and the second flared portion 524.

The third implant segment 608 can have a third elongated body 624 with a sixth terminating end 626 (e.g., a first distal end) defined by the second break 622. The sixth terminating end 626 can define a first distal end of the third elongated body 624, and a second distal end can be defined by the second terminating end 328 of the implant 110. The third implant segment 608 can further include one geometric protrusion 202 extending from the first side 208 and one geometric protrusion 202 (e.g., in the trapezoidal pyramid configuration 502) extending from the second side 210.

Various implant segments of the implant 110 can correspond to various portions (e.g., features, curves, contours, and/or shapes) of bone sections being fused by the implant 110. For instance, a shape and/or depth dimension 236 of the first implant segment 604 can correspond to and align with the manubrium 526 of the sternum 108. A shape and/or a depth dimension 236 of the second implant segment 606 can correspond to and align with the sternum body 528 of the sternum 108. A shape and/or depth dimension 236 of the third implant segment 608 can correspond to and align with a xiphoid process 628 of the sternum 108.

In some examples, the implant 110 can be measured to be placed and run the length of the entire sternum 108, for example from the jugular (suprasternal) notch of the manubrium to the distal tip of the xiphoid process. In other instances, implant 110 can be measured and fit into place along the inside edges of the manubrium only. Additionally, the implant 110 can be measured and fit into place along the inside edges of the manubrium, through the manubriosternal joint or second costal notch and into the sternum body 528 only. In some examples, the implant 110 can be measured and fit into place along the inside edges of the manubrium 526, through the manubriosternal joint or second costal notch and into the sternum body 528, including the third, fourth, fifth, and sixth costal notches of the sternum 108 to the distal tip of the sternum body 528 only and on through the seventh costal notch and through the body of the xiphoid process 628 or to the distal tip of the xiphoid process 628.

Furthermore, it is to be understood that the shapes of features disclosed herein (e.g., triangle, square, rectangle, trapezoid, etc.) may include rounded or slightly rounded corners and/or do not necessarily require perfectly adjoining lines or sharp angles to be defined as the aforementioned shapes. In other words, the features may substantially resemble the shapes discussed herein without being perfectly shaped.

In some examples, the implant 110 depicted in FIGS. 2-6 (and throughout this disclosure) is comprised of various synthetic and/or natural materials such as a demineralized cortical bone graft, for example harvested from a donor or from the recipient's own bone cortical bone fibers, cancellous bone fibers, collagen sponge, cortical bone graft, synthetic bone, and/or tissue graft, and/or combinations thereof. The implant 110 can include a spun bone wool sternal gasket harvested from human tissue. The implant 110 can be made from cortical bone, cancellous bone, dense cortical/cancellous bone, collagen fibers, and/or any number of other suitable human or animal bone derived tissues. In at least one example, the implant 110 can include at least one cellular growth factor. The cellular growth factors can include at least one of the following: bone morphogenetic proteins (BMPs), mesenchymal stem cells, blood, osteoclasts, osteoblasts, antibiotics, analgesics, and/or medications. In at least one example, blood can be drawn from the patient, spun down, and added or soaked into implant 110 so that the implant 110 becomes a carrier for the patient's own cells. The synthetic materials can as synthetic tissue, vicryl, polypropylene, stainless steel, titanium, polyether ether ketone (PEEK), polyetherketone (PEK), polymers, metals, poly (methyl methacrylate) (PMMA) or other synthetic materials and/or suture materials. The elongated body 206 and the bone anchors 116 can be formed of a same material and/or different materials.

In at least one example, the implant 110 can be porous and/or fibrous to receive at least one cellular growth factor. The porous and/or fibrous material may allow blood and other fluids to easily pass through the bone fibers. This allows bone healing cells, osteoclasts and osteoblasts, naturally occurring bone morphogenic proteins and other cells, and/or added osteoconductive substances to incorporate easily throughout the implant 110. Additionally, the inclusion of a cellular growth factor can provide for significantly quicker and greater rates of healing and fusion. The cellular growth factors (e.g., and any other carrier components) can be soaked into the implant 110 prior to compressing the bone sections together with the implant 110 in between, or inserted through the one or more ports 134 after compressing and/or securing the implant 110 and bone sections.

Cellular growth factors and/or other carrier components held by the implant 110 can include one or more of bone growth-promoting material, bone morphogenetic proteins, mesenchymal stem cells, blood, osteoclasts, osteoblasts, antibiotics, analgesics, plasma rich protein (PRP), bone marrow aspirate (BMA), saline, antibiotics, analgesics, anticoagulants, bone growth factors such as bone morphogenetic proteins (BMPs), hydroxyapatite, hyaluronic acids, beta-tricalcium phosphate (BTCP), surgical glues utilizing natural materials as well as human and/or animal tissues, demineralized bone matrix (DBM) powders, particulates, silicates, pastes, putties, cancellous chips, allograft tissues, xenograft tissues, collagen fibers, collagen matrix, collagen thrombin, hemostatic medicine, and/or medications or materials a surgeon finds appropriate to use in the treatment of the patient. The fibrous nature of the implant 110 may allow fluids and substances such as the carrier component to get caught up, interdigitated, engorged, and locked into the implant 110.

The fibrous nature of the implant 110, such as spun bone, can also function to fill voids and also give flexibility and compressive characteristics to the implant 110. The fibrous nature of the implant 110 may provide a porous characteristic to allow blood and fluid to penetrate, wick into, pass through, and become absorbed by the implant 110. As such, the implant 110 may become engorged with blood or other fluids and act as a clot against the cut and bleeding of the cut sternum 108. The system 100, accordingly, can act as a hemostasis device to help cause the incised bone to stop bleeding. Usage of the cancellous bone manipulator tool 114 and the bone clamp 112 to complete the bone fusing process can further improve the hemostasis properties of the system 100.

The porous nature of the implant 110 can also provide flexibility for aligning the implant 110 with the cut bone sections. Thus, the implant 110 can have the ability to adapt easily to the natural contours of the body. Flexibility also can allow the implant 110 to overcome challenging bone structure either created by the surgeon upon accessing the chest cavity during entry or as a result of trauma, loss of bone, and/or other naturally occurring issues. Furthermore, as discussed in greater detail below, the bone material can be moved, scraped, and otherwise manipulated with the cancellous bone manipulator tool 114 to further adapt the receiving portion of the cut bone sections to match a shape of the implant 110 (e.g., the geometric protrusions 202).

Any of the benefits or advantages of the implant 110 discussed herein can result from a particular thickness, dimension, material composition, or combinations thereof of the implant 110, in addition to other aspects of the disclosed technology.

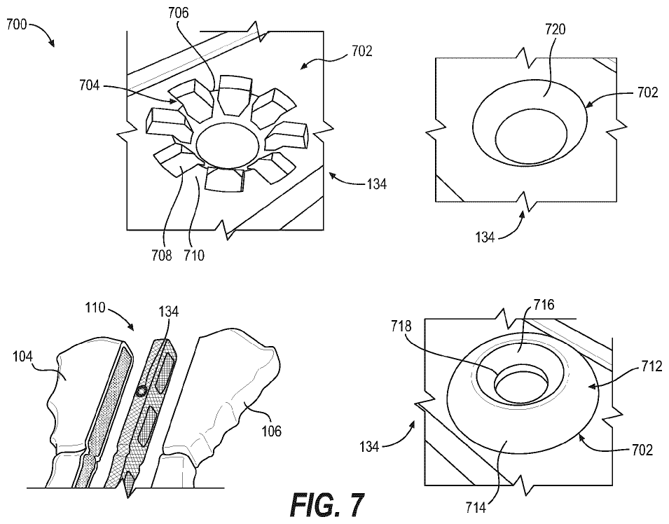

Turning to FIG. 7, an example system 700 includes the one or more ports 134 of the implant 110, which can form at least a portion of the system 100. FIG. 7 illustrates multiple embodiments of the one or more ports 134.

The one or more ports 134 can include one or more tactile identifiers 702. For instance, the port 134 can include a serrated edge 704 as the tactile identifier 702. The serrated edge 704 has circumference 706 with a plurality of alternating idented portions 708 or slits and raised portions 710 located around the port 134. For instance, the port 134 can be circular with the idented portions 708 and the raised portions 710 arranged extending radially and/or around the circumference of the port 134. In some instances, the port 134 includes a raised lip 712 as the tactile identifier 702. The raised portions 710 can include an outer curved wall 714 extending from the front side 212 and an inner curved wall 716 extending from an inner edge 718 of the port 134. The outer curved wall 714 can have a convex contour or curve and the inner curved wall 716 can have a concave contour or curve. Finally, in some instances, the tactile identifier 702 is a dip or lowered portion 720 arranged circumferentially around the port 134. The dip or lowered portion 720 can include a concave contour/curved surface.

In some examples, the tactile identifier 702 provides tactile or haptic feedback so the port 134 can be located tactilely-that is, by using fingers or touch. As such, the port 134 may be identifiable by the surgeon or other medical personnel easily and quickly without needing a visual line-of-sight to the port 134. Because the tactile identifier 702 can be formed of a material that substantially maintains a shape (e.g., a flexible and/or at least partially rigid material), the port 134 can be located by the surgeon and used after the implant 110 has been fully compressed and secured between the first bone section 104 and the second bone section 106. As such, carrier components can be inserted (e.g., injected) into the port 134 at various stages minutes, hours, or even days, after compressing and securing the implant 110 between the first bone section 104 and the second bone section 106. This type of treatment has significant advantages by improving patient outcomes while reducing hospital outpatient time.

In some instances, the port 134 has an inverted conical shape opening on the front side 212 and/or the raised surface 232 of the implant 110. The port 134 can have a shape that facilitates injections of materials directly into the implant 110 either preoperatively, during the procedure or post operatively through the skin. Moreover, an incision into the skin can also be made post operatively to facilitate the injection into the port 134. The port 134 can be inset with tantalum or another radio opaque material to aid the surgeon in identifying and/or locating the port 134 post operatively (e.g., with fluoroscopic assistance).

The port 134 can be made of similar or identical material as the implant 110. The port 134 can be formed of cortical, cancellous, dense cancellous, and/or fiber bone material. Additionally or alternatively, the port 134 can be formed of demineralized bone matrix (DBM) and/or titanium, Polyether ether ketone (PEEK), polyetherketoneketone (PEKK), dense cortical bone, fibrin glue, collagen, stainless steel, surgical plastics of all manner, beta tricalcium phosphate (TCP), coral, silicate, hydroxy appetite (HA), materials containing peptide additives, silicone, xenograft, autograft, allograft, and other manmade or natural materials. Like the implant 110, the port can be made form bone or tissue of the patient receiving the implant 110. In some examples, a porosity of the port and/or surrounding graft tissue can be in a range from 30% to 80% nano, micro, and milli porosity. Although the port 134 is depicts as forming part of the implant 110, it is to be understood that the port 134 my form a part of other medical apparatuses and/or implants to provide an injection pathway into the body of the patient. For instance, the port 134 may be formed into an allograft, an autograft, a xenograft, or any other device used for surgical repairs.

FIG. 8 depicts an example system 800 including the bone clamp 112 for fusing two sections of bone, which can form at least a portion of the system 100. The bone clamp 112 can include the single-handed handle arrangement 118 to open and close the first gripping arm 128 and the second gripping arm 130 using only one hand.

The single-handed handle arrangement 118 can include the shaft 120 with the spring-loaded slider 122 slidably coupled to the shaft 120. The first handle 124 extends from a first distal end 802 of the shaft 120 and the first gripping arm 128 extends from a second distal end 804 of the shaft 120. The first handle 124 and the first gripping arm 128 can extend perpendicularly or substantially perpendicularly from the shaft 120. Moreover, the first handle 124 and the first gripping arm 128 can extend in a same direction or, as depicted in FIG. 8, extend in different or opposing directions (e.g., with the first handle 124 extending upward and the first gripping arm 128 extending downward).

The single-handed handle arrangement 118 of the bone clamp 112 can further include the second handle 126 extending from a first distal end 806 of the spring-loaded slider 122. The second handle 126 can extend from a portion of the spring-loaded slider 122 that includes a first spring stop 808 and/or a first shaft coupler 810. The first spring stop 808 is a portion of the spring-loaded slider 122 that abuts a first end 812 of the spring 132, for instance, with an inner stop surface 814. The first shaft coupler 810 is a portion of the spring-loaded slider 122 coupling the spring-loaded slider 122 to the shaft 120 (e.g., a loop). In some examples, the first shaft coupler 810 can be a rail attachment or protrusion to mate with a channel in the shaft 120 or any other mechanism to slidably couple the spring-loaded slider 122 to the shaft 120. The second gripping arm 130 can extend from a second distal end 816 of the spring-loaded slider 122. The second gripping arm 130 extends in a first same direction as the first gripping arm 128 and the second handle 126 extends in a second same direction as the first handle 124, which is a different or opposing direction as the first same direction. Moreover, a second spring stop 818 can be formed by a second inner stop surface 820 of a second shaft coupler 822 disposed at the second distal end 816 to abut a second end of the spring 132. The second gripping arm 130 can also extend from the second shaft coupler 822. A bar 824 can connect the first shaft coupler 810 to the second shaft coupler 822 by attached to and/or extending from the first shaft coupler 810 and the second shaft coupler 822. The bar 824 can extend substantially parallel to the shaft 120 and the spring 132 and define a body of the spring-loaded slider 122. The second handle 126, the first shaft coupler 810, and a first end of the bar 824 can intersect at a juncture 826, and a second end of the bar 824 can mate to a first side of the second shaft coupler 822 with the second gripping arm 130 extending from a second side of the second shaft coupler 822 opposite the first side.

In some examples, the first handle 124 can include a first handle extender 828 and a second handle extender 830. The first handle extender 828 and the second handle extender 830 extend perpendicularly from a distal end of the first handle 124, such that the first handle 124, the first handle extender 828, and the second handle extender 830 form a “T” shape. Similarly, the second handle 126 can include a third handle extender 832 and a fourth handle extender 834 also forming a “T” shape. The first handle extender 828, the second handle extender 830, the third handle extender 832, and/or the fourth handle extender 834 can be circular and/or rollers and may be formed of a similar material or different material as other components of the bone clamp 112 (e.g., hard rubber, plastic, metal, foam, etc.). Moreover, the handle extenders can form a detachable connection with the first handle 124 and the second handle 126 to be easily snapped on and off for storing the bone clamp 112. The entire spring-loaded slider 122 unit (e.g., including the second handle 126, the first handle extender 828, the second handle extender 830, the first shaft coupler 810, the second shaft coupler 822, and the second gripping arm 130) can be disposed between the first distal end 802 and the second distal end 804 of the shaft 120.

The single-handed handle arrangement 118 can include a spacing distance 836 between the first handle 124 and second handle 126 such that both the first handle 124 and the second handle 126 can be grabbed by one hand (e.g., with a thumb wrapping around the first handle extender 828 or the second handle extender 830 of the first handle 124, and other fingers wrapping around the third handle extender 832 and fourth handle extender 834 of the second handle 126). Moreover, the components of the bone clamp 112 are arranged so that the bone clamp 112 is in a closed position (e.g., with the first gripping arm 128 and the second gripping arm 130 brought together a first distance) when the bone clamp 112 is in a resting state (e.g., with the spring 132 extended). A surgeon or other medical personnel can use the single-handed handle arrangement 118 to pull the bone clamp 112 open with one hand (e.g., while the other hand is holding the implant 110 in place). This causes the bone clamp to be in an open position with the first gripping arm 128 and the second gripping arm 130 pulled apart a second distance that is greater than the first distance. The spring 132 is compressed when the bone clamp 112 in the open position. While holding the bone clamp 112 open with one hand by pulling the first handle 124 and the second handle 126 together, the bone clamp 112 can be positioned around the first bone section 104, the implant 110, and the second bone section 106 so that the first gripping arm 128 abuts the first bone section 104 and a second gripping arm 130 abuts the second bone section 106. The surgeon closes the bone clamp 112 by releasing the first handle 124 from the second handle 126 and letting the spring 132 decompress, pushing the spring-loaded slider 122 along the shaft 120 so the first gripping arm 128 and the second gripping arm 130 are brought back together. This single-handed handle arrangement 118 significantly improves the ability for the surgeon to align the implant 110 with the cut bone sections while opening and closing the bone clamp 112 to compress the two sections of bone together.

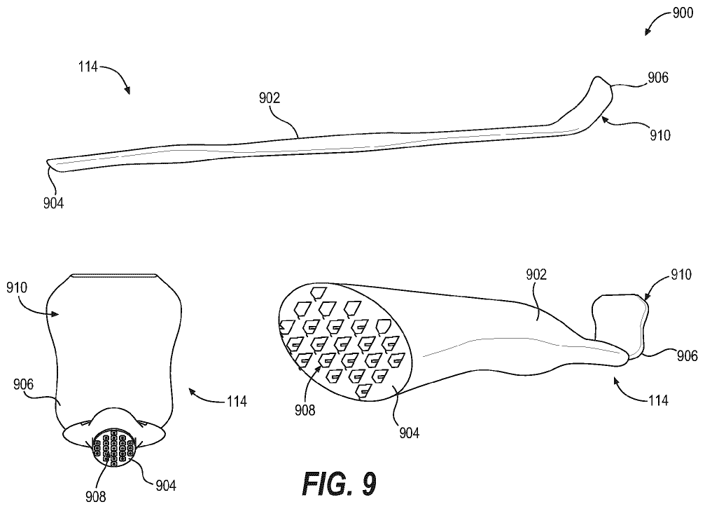

FIG. 9 illustrates an example system 900 including the cancellous bone manipulator tool 114 for fusing two sections of bone, which can form at least a portion of the system 100.

In some examples, the cancellous bone manipulator tool 114 includes an elongated gripping portion 902 with a first distal end 904 and a second distal end 906. The first distal end 904 can terminate with a scraper surface 908 which forms, in some examples, a non-perpendicular angle with the elongated gripping portion 902. The scraper surface 908 can have one or more deformations or scraping elements, such as protrusions, cuts, sand, indents, pointed edges, combinations thereof, and the like. The scraper surface 908 can be used to roughen the incised sternal edges of the first bone section 104 and/or the second bone section 106, which can help the implant 110 grip the incised sternal edges and improve performance of the system 100 for achieving successful patient outcomes.

The second distal end 906 of the cancellous bone manipulator tool 114 can terminate as or include a curved paddle 910. The curved paddle 910 can bend away from an axis defined by the elongated gripping portion 902 and have a greater or flared width relative to a width of the elongated gripping portion 902. In other words, the curved paddle 910 can be a flatted end of the elongated gripping portion 902. In some examples, the surgeon uses the curved paddle 910 to press into or scrape the cancellous tissue of the first bone section 104 and/or the second bone section 106. For instance, the surgeon can use the curved paddle 910 (and/or the scraper surface 908) to form an indentation or impression into the cancellous tissue at least partially corresponding to a shape of the geometric protrusions 202. Accordingly, the curved paddle 910 can be used to improve the ability of the bone anchors 116 to fit into and mate with the cancellous tissue. When this occurs, the blood flow from the cancellous tissue into the porous/fibrous material of the implant 110 is increased, further improving integration of the implant into the healing bone tissue and ultimately improving patient outcomes.

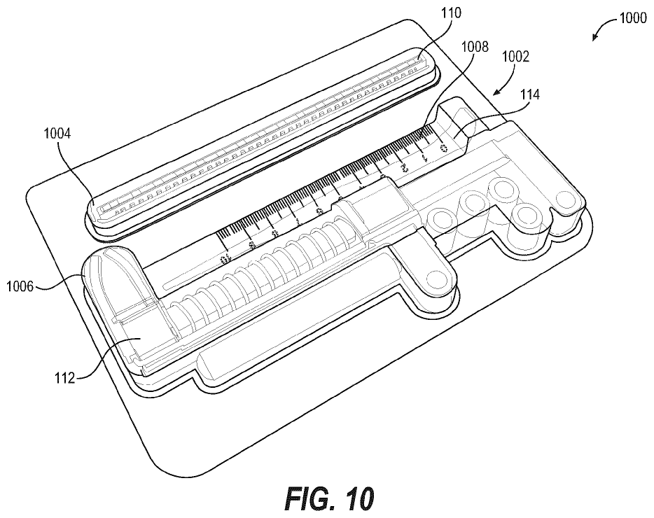

FIG. 10 illustrates an example system 1000 including the bone fusing medical kit 102 for fusing two sections of bone, which can form at least a portion of the system 100. The bone fusing medical kit 102 can integrate the implant 110, the bone clamp 112, and the cancellous bone manipulator tool 114 into a single unit or product in a self-contained, sterile package (e.g., to form an “integrated” bone fusing medical kit 102).

For instance, the bone fusing medical kit 102 can include one or more holding areas 1002 formed as impressions into a holding material to hold the implant 110, the bone clamp 112, and/or the cancellous bone manipulator tool 114 in place. A first holding area 1004 can have a first shape corresponding to the implant 110 and can secure the implant 110 in the medical kit 102. A second holding area 1006 can have a second shape corresponding to the bone clamp 112 and can secure the bone clamp 112 in the medical kit 102. Additionally, a third holding area 1008 can have a third shape corresponding to the cancellous bone manipulator tool 114 and can secure the cancellous bone manipulator tool 114 in the bone fusing medical kit 102. In other words, the one or more holding areas 1002 can be imprints with particular shapes formed into the holding material (e.g., a sterile plastic) that align with and mate with the implant 110, the bone clamp 112, and/or the cancellous bone manipulator tool 114 to form the bone fusing medical kit 102.

As such, the sterile surgical packaging can keep the components of the bone fusing medical kit 102 stored in a sterile, sealed, and protected environment prior to use. Moreover, the bone fusing medical kit 102 keeps the tools for fusing two sections of bone (e.g., the implant 110, the bone clamp 112, and/or the cancellous bone manipulator tool 114) in close proximity to each other. The bone fusing medical kit 102 can additionally or alternatively include a syringe/needle for injecting the carrier component into the port 134, and/or other tools for performing the bone fusing procedure.

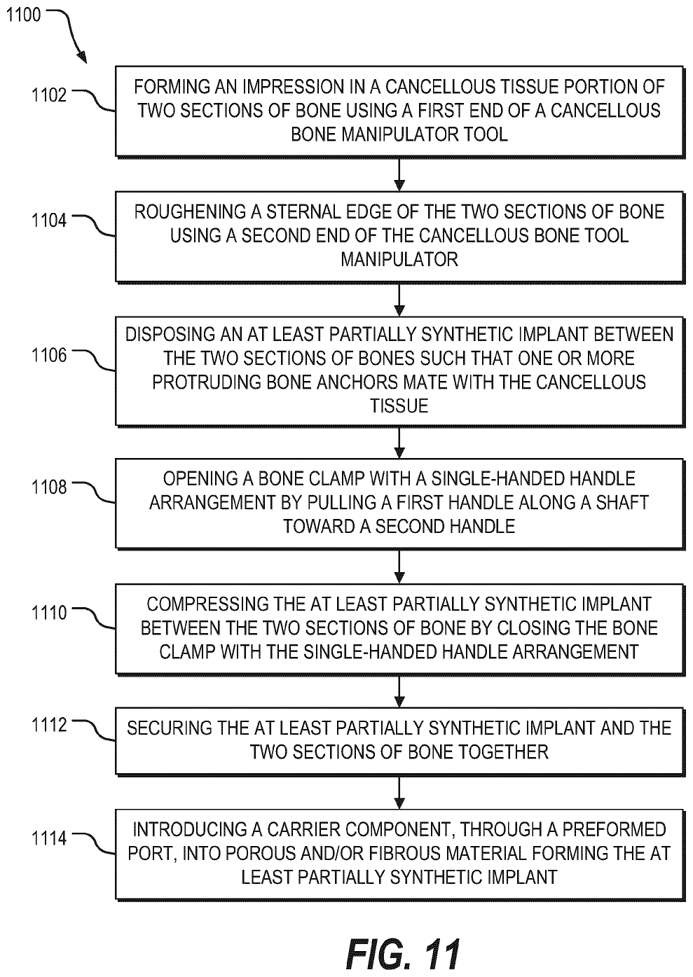

FIG. 11 illustrates an example method 1100 for fusing two sections of bone that can be performed by at least the system 100. At operation 1102, the method 1100 forms an impression in a cancellous tissue portion of two sections of bone using a first end of a cancellous bone manipulator tool. At operation 1104, the method 1100 roughens a sternal edge of the two sections of bone using a second end of the cancellous bone tool manipulator. At operation 1106, the method 1100 disposes an at least partially synthetic implant between the two sections of bone such that one or more protruding bone anchors mate with the cancellous tissue. At operation 1108, the method 1100 opens a bone clamp with a single-handed handle arrangement by pulling a first handle along a shaft toward a second handle. At operation 1110, the method 1100 compresses the at least partially synthetic implant between the two sections of bone by closing the bone clamp with the single-handed handle arrangement. At operation 1112, the method 1100 secures the at least partially synthetic implant and the two sections of bone together. At operation 1114, the method 1100 introduces a carrier component, through a preformed port, into porous and/or fibrous material forming the at least partially synthetic implant.

It is to be understood that the specific order or hierarchy of steps in the method depicted in FIG. 11 (and other methods disclosed herein) are instances of example approaches and can be rearranged while remaining within the disclosed subject matter. For instance, any of the operations depicted in FIG. 11 can be omitted, repeated, performed in parallel, performed in a different order, and/or combined with any other of the operations depicted in FIG. 11. For instance, operation 1114 can but does not necessarily occur subsequently to operation 1112. Moreover, any of the systems or methods illustrated in FIGS. 1-11 can be combined together and/or form at least a portion of the system 100.

Moreover, while the disclosure is generally focused on the implant 110 being used to fuse the first bone section 104 and the second bone section 106 of the sternum 108 back together, for example following an open sternotomy procedure, the implant 110 can be used in other parts of the human body to aid in fusion, including, but not limited to the ankle, foot, knee, spine, hip and SI joints, shoulder, long bones, elbow, cranium, maxillofacial repair, and the like without deviating from the scope of this disclosure.

While the present disclosure has been described with reference to various implementations, it will be understood that these implementations are illustrative and that the scope of the present disclosure is not limited to them. Many variations, modifications, additions, and improvements are possible. More generally, implementations in accordance with the present disclosure have been described in the context of particular implementations. Functionality may be separated or combined differently in various implementations of the disclosure or described with different terminology. These and other variations, modifications, additions, and improvements may fall within the scope of the disclosure as defined in the claims that follow.

What is claimed is:

1. A system to fuse two sections of a bone, the system comprising:
an at least partially synthetic implant including:
an elongated body;
first one or more bone anchors at a first side of the elongated body; and
second one or more bone anchors at a second side of the elongated body; and
a bone clamp with a single-handed handle arrangement.

2. The system of claim 1, further comprising,
an elongated cancellous bone manipulator tool with a curved paddle at a first manipulator end and a scraper surface at a second manipulator end.

3. The system of claim 2,
wherein,
the at least partially synthetic implant, the bone clamp, and the elongated cancellous bone manipulator tool form an integrated medical kit.

4. The system of claim 1,
wherein,
the single-handed handle arrangement includes:
a shaft defining a length of the bone clamp with a first handle extending from the shaft; and
a spring-loaded slider slidably coupled to the shaft with a second handle extending from the spring-loaded slider.

5. The system of claim 4,
wherein,
the first handle extends from a first shaft end,
the second handle extends from a first slider end, and
the bone clamp further includes,
a first gripping arm extending from a second shaft end opposite the first shaft end; and
a second gripping arm extending from a second slider end opposite the first slider end.

6. The system of claim 5,
wherein,
the first gripping arm and the second gripping arm are maintained in a closed position by a spring when the bone clamp is in a resting state.

7. The system of claim 1,
wherein,
the first one or more bone anchors and the second one or more bone anchors are a plurality of geometric protrusions operable to mate with cancellous tissue of the two sections of bone.

8. The system of claim 7,
wherein,
the plurality of geometric protrusions have a triangular lateral cross-section profile and a trapezoidal longitudinal cross-section profile.

9. The system of claim 1,
wherein,
the elongated body includes a plurality of implant sections with a plurality of length dimensions corresponding to a plurality of particular bones.

10. The system of claim 9,
wherein,
the plurality of particular bones include a manubrium, a sternum body, and a xiphoid process.

11. A system to fuse two sections of a bone, the system comprising:
an at least partially synthetic implant including:
an elongated body;
a first bone anchor extending from a first side of the elongated body as a first geometric protrusion; and
a second bone anchor extending from a second side of the elongated body as a second geometric protrusion; and
a bone clamp to compress the at least partially synthetic implant between the two sections of bone.

12. The system of claim 11,
wherein,
the first geometric protrusion is shaped to press into first cancellous tissue of a first section of the two sections of the bone, and
the second geometric protrusion is shaped to press into second cancellous tissue of a second section of the two sections of bone.

13. The system of claim 11,
wherein,
the bone clamp has a single-handed handle arrangement including:
a shaft with a spring-loaded slider slidably coupled to the shaft,
a first handle extending from the shaft, and
a second handle extending from the spring-loaded slider.

14. The system of claim 11,
wherein,
the at least partially synthetic implant is formed of a porous and/or fibrous material operable to receive a carrier component, and
the at least partially synthetic implant has a port to receive the carrier component, the port being disposed on a front surface of the elongated body between the first side and the second side.

15. The system of claim 14,
wherein,
the port includes a tactile identifier.

16. The system of claim 15,
wherein,
the tactile identifier is a raised or serrated lip around the port.

17. A method to fuse two sections of a bone, the method comprising:
forming an impression in a cancellous tissue portion of the two sections using an end of a cancellous bone manipulator tool;
disposing an at least partially synthetic implant between the two sections of the bone, the at least partially synthetic implant including one or more protruding bone anchors to mate with cancellous tissue of the two sections of the bone; and
compressing the at least partially synthetic implant between the two sections using a bone clamp with a single-handed handle arrangement.

18. The method of claim 17,
wherein,
the single-handed handle arrangement includes:
a first handle spaced apart from a second handle,
the first handle extends from a shaft of the bone clamp, and
the second handle extends from a spring-loaded slider of the bone clamp slidably coupled to the shaft, and
using the bone clamp includes pulling the first handle towards the second handle.

19. The method of claim 17, further comprising,
securing the at least partially synthetic implant and the two sections of bone together, and
introducing, subsequent to securing the at least partially synthetic implant and the two sections of bone together, a carrier component into porous and/or fibrous material forming the at least partially synthetic implant.

20. The method of claim 19,
wherein,
introducing the carrier component into the porous and/or fibrous material includes introducing the carrier component through a preformed port disposed on the at least partially synthetic implant.
